(19) United States
(12) Patent Application Publication
(10) Pub. No.: US 2011/0015713 A1
(43) Pub. Date: Jan. 20, 2011

(54) SYSTEMS AND METHODS FOR REDUCING LEAD HEATING AND THE RISKS OF MRI-INDUCED STIMULATION

(75) Inventor: Xiaoyi Min, Thousand Oaks, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/891,602

(22) Filed: Sep. 27, 2010

Related U.S. Application Data
(63) Continuation-in-part of application No. 12/257,263, filed on Oct. 23, 2008.

(51) Int. Cl. A61N 1/05

(52) U.S. Cl. 607/116

(57) ABSTRACT

An implantable medical lead is described herein wherein the lead includes a tubular body, an electrode, a lead connector end and a helical conductor. The tubular body includes a proximal end and a distal end. The electrode is coupled to the body near the distal end. The lead connector end is coupled to the body near the proximal end. The helical conductor coil extends through the body from the lead connector end to the electrode. In extending through the body, the helical conductor coil first extends distally for a distance, then proximally for the distance, and then distally for the distance within a single helical layer of the helical conductor coil. The electrode may be a ring electrode.

### CROSS-REFERENCE TO RELATED APPLICATIONS

This application is a continuation-in-part (“CIP”) application of U.S. patent application Ser. No. 12/257,263, filed Oct. 23, 2008, entitled “Systems and Methods for Exploiting the Tip or Ring Conductor of an Implantable Medical Device Lead During an MRI to Reduce Lead Heating and the Risks of MRI-Induced Stimulation” (Attorney Docket A08P1048), and incorporated by reference herein in its entirety.

### FIELD OF THE INVENTION

The invention generally relates to leads for use with implantable medical devices, such as pacemakers or implantable cardioverter-defibrillators (ICDs), and to techniques for reducing tip heating within such leads during a magnetic resonance imaging (MRI) procedure.

### BACKGROUND OF THE INVENTION

MRI is an effective, non-invasive magnetic imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed radio-frequency (RF) magnetic field is then applied causing the protons to begin to precess around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

However, MRI procedures are problematic for patients with implantable medical devices such as pacemakers and ICDs. A significant problem is that the strong fields of the MRI can induce currents within the lead system that cause the electrodes of leads of the implantable device to become significantly heated, potentially damaging adjacent tissues or the lead itself. Heating is principally due to the RF components of the MRI fields. In worst-case scenarios, the temperature at the tip of an implanted lead can increase as much as 70 degrees Celsius (C) during an MRI. Although such a dramatic increase is probably unlikely within a system wherein leads are properly implanted, even a temperature increase of only about 8°-13° C. can cause myocardial tissue damage. Furthermore, any significant heating of the electrodes of pacemaker and ICD leads, particular tip electrodes, can affect pacing and sensing parameters associated with the tissue near the electrode, thus potentially preventing pacing pulses from being properly captured within the heart of the patient and/or preventing intrinsic electrical events from being properly sensed by the device. The latter may potentially result, depending upon the circumstances, in therapy being improperly delivered or improperly withheld. Another significant concern is that any currents induced in the lead system can potentially generate voltages within cardiac tissue comparable in amplitude and duration to stimulation pulses and hence might trigger unwanted contractions of heart tissue. The rate of such contractions can be extremely high, posing significant clinical risks on patients.

Hence, there is a need to reduce heating in the leads of implantable medical devices, especially pacemakers and ICDs, and to also reduce the risks of improper tissue stimulation during an MRI, which is referred to herein as MRI-induced pacing.

### SUMMARY OF THE INVENTION

In accordance with various exemplary embodiments of the invention, a lead is provided for use with an implantable medical devices for implant within a patient wherein the lead includes first and second electrodes for placement adjacent patient tissues, an inner conductor for routing signals along the lead between the first electrode and the implantable medical device, and an outer conductor for routing signals along the lead between the second electrode and the implantable medical device. An insulator is interposed between the outer conductor and patient tissues. An inductive element is connected along the outer conductor between the second electrode and the implantable medical device. The inductive element is configured to act as a band stop filter at the RF of an MRI, converting the outer conductor into a floating shield or electromagnetic signal shield to, e.g., shield the inner conductor during an MRI.

In one embodiment, an implantable medical lead is described herein wherein the lead includes a tubular body, an electrode, a lead connector end and a helical conductor. The tubular body includes a proximal end and a distal end. The electrode is coupled to the body near the distal end. The lead connector end is coupled to the body near the proximal end. The helical conductor coil extends through the body from the lead connector end to the electrode. In extending through the body, the helical conductor coil first extends distally for a distance, then proximally for the distance, and then distally for the distance within a single wound layer of the helical conductor coil. The electrode may be a ring electrode.

The helical conductor coil may include multiple filars. In the helical conductor coil first extending distally for a distance, then proximally for the distance, and then distally for the distance, the multiple filars may first extend distally for the distance, then proximally for the distance, and then distally for the distance, the filars forming a double back type of pattern within the single wound layer of the helical conductor coil.

The lead may also include at least one band stop filter coupled to the helical conductor coil and located between the lead connector end and the electrode. In such an embodiment, the filars may form a first double back pattern between the lead connector end and the band stop filter and a second double back pattern between the band stop filter and the electrode.

The lead may also include multiple band stop filters coupled to the helical conductor coil and located between the lead connector end and the electrode. In such an embodiment, the filars may form a double back pattern between a pair of band stop filters. The band stop filters may be spaced apart from each other along the tubular body at a distance of approximately a quarter wavelength.

In one embodiment, the multiple filars includes a first number of filars, a second number of filars and a third number of filars. The first number of filars extend distally for the distance and join to a distal end of the second number of filars, the second number of filars then extends proximally for the distance and join to a proximal end of the third number of filars, the third number of filars then extends distally, the filars forming a double back type of pattern within the single wound layer of the helical conductor coil. The first number of filars may be one, two or more filars.

In one embodiment, at least one of the filars of the multiple filars are insulated and at least another of the filars of the multiple filars are uninsulated, there being a pattern of uninsulated to insulated filars in the helical conductor coil. Examples of possible patterns of uninsulated to insulated filars includes: 2 to 1; 2 to 2; 3 to 1; 3 to 2; 3 to 3; 4 to 1; 4 to 2; or etc. In some embodiments, at least some of the filars include electrically conductive portions formed of MP35N.

In another embodiment, an implantable medical lead is described herein wherein the lead includes a tubular body, an electrode, a lead connector end and a helical conductor. The tubular body includes a proximal end and a distal end. The electrode is coupled to the body near the distal end. The lead connector end is coupled to the body near the proximal end. The helical conductor coil extends through the body from the lead connector end to the electrode and is configured to cause current to double back along at least a portion of the length of the helical conductor coil within a single wound layer of the helical conductor coil.

In another embodiment, a method of manufacturing an implantable medical lead is described herein wherein the method includes: providing a tubular body; coupling an electrode to a distal portion of the tubular body and a lead connector end to a proximal portion of the tubular body; and forming a helical conductor coil extending through the body from the lead connector end to the electrode, the coil being configured to cause current to double back along at least a portion of the length of the helical conductor coil within a single wound layer of the helical conductor coil.

In yet another embodiment, any of the aforementioned double back (i.e., zig-zag) arrangements discussed above in the context of a helically wound multi-filar conductor coil may be applied to a helically twisted multi-filar conductor cable.

In still another embodiment, a method of assembling an implantable medical lead includes: helically co-winding multiple filars into a single helical layer; electrically joining a distal end of a first group of one or more of the multiple filars to a distal end of a second group of one or more of the multiple filars; electrically joining a proximal end of the second group of one or more of the multiple filars to a proximal end of a third group of one or more of the multiple filars; electrically coupling a proximal end of the first group of one or more of the multiple filars to an electrical contact of a lead connector end; and electrically coupling a distal end of the third group of one or more of the multiple filars to a distal electrode. Depending on the version of the embodiment, the single helical layer may include part of a helically wound multi-filar coil conductor or part of a helically wound multi-filar cable conductor.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a stylized representation of an MRI system along with a patient with a pacer/ICD implanted therein with leads employing ring electrode inductor (band stop filter) elements;

FIG. 2 is a block diagram, partly in schematic form, illustrating a bipolar lead for use with the pacer/ICD of FIG. 1 wherein an inductor (band stop filter) element is mounted to the lead near the ring electrode, and also illustrating a pacer/ICD connected to the lead;

FIGS. 3A and 3B are, respectively, an elevation view of a portion of the bipolar lead of FIG. 2 and a cross-sectional view as taken along section line 3B-3B of FIG. 3A;

FIG. 4 is a block diagram, partly in schematic form, illustrating a bipolar lead for use with the pacer/ICD of FIG. 1 wherein an inductor (band stop filter) element is mounted to the lead in the header of the lead, and also illustrating a pacer/ICD connected to the lead;

FIG. 5 is an elevational view of a portion of the bipolar lead of FIG. 4, particularly illustrating the placement of a inductor element in the header of the lead;

FIG. 6 is a block diagram, partly in schematic form, illustrating a bipolar lead for use with the pacer/ICD of FIG. 1 wherein inductor (band stop filters) elements are mounted to the lead in both the header of the lead and near the ring electrode, and also illustrating a pacer/ICD connected to the lead;

FIG. 7 is a block diagram, partly in schematic form, illustrating a bipolar lead for use with the pacer/ICD of FIG. 1 wherein inductor (band stop filters) elements are mounted to the lead in both the header of the lead and near the ring electrode for each of the ring conductor and the tip conductor, and also illustrating a pacer/ICD connected to the lead;

FIG. 8 is a block diagram, partly in schematic form, illustrating a bipolar lead for use with the pacer/ICD of FIG. 1 wherein inductor (band stop filters) elements are mounted to the lead in the header of the lead, near the ring electrode and there between at generally even spacing, for example, at distances of a quarter wavelength, and also illustrating a pacer/ICD connected to the lead;

FIG. 9 is a longitudinal cross section of a distal end of an active fixation lead;

FIG. 10 is a longitudinal cross section of a distal end of an passive fixation lead;

FIGS. 11A-11C are longitudinal cross section segments of a lead body similar to that depicted in FIG. 9, wherein the filars of the ring conductor are in various patterns of insulated and un-insulated;

FIG. 12 is a schematic diagram of a lead;

FIG. 13 is a longitudinal cross section of a ring conductor with the rest of the lead depicted in phantom lines;

FIG. 14 is a longitudinal cross section of a ring conductor with the rest of the lead depicted in phantom lines, wherein the ring conductor combines the concepts depicted in the leads of FIGS. 8, 12 and 13; and

FIG. 15 is a simplified, partly cutaway view, illustrating the pacer/ICD of FIG. 1 along with a more complete set of leads implanted in the heart of the patient, wherein the RV lead includes an inductor element or band stop filter near the location of the ring electrode.

### DETAILED DESCRIPTION OF THE PREFERRED EMBODIMENTS

The following description includes the best mode presently contemplated for practicing the invention. The description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of MRI System

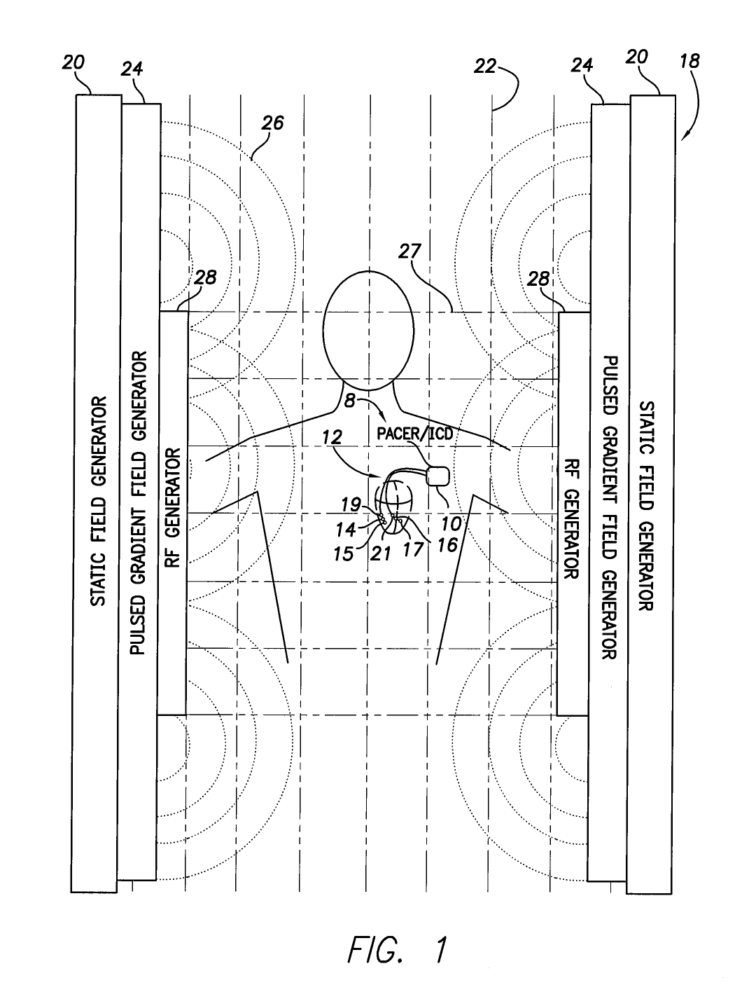

FIG. 1 illustrates an implantable medical system 8 having a pacer/ICD 10 for use with a set of coaxial bipolar pacing/sensing leads 12, which include tip and ring electrodes 14, 15, 16 and 17, as well as ring electrode inductor elements 19 and 21. The inductor elements 19 and 21 act as band stop filters at the RF (e.g., 64 MHz or 128 MHZ) of the MRI to substantially reduce, if not entirely prevent, current being induced via the MRI magnetic fields in the conductors leading to the ring electrodes, thereby reducing MRI induced heating of the ring electrodes. In some embodiments, the inductor elements 19 and 21 acting as band stop filters at the RF of the MRI results in the outer ring conductors of the leads (not separately shown within FIG. 1) to turn into floating outer coils that act as RF shields for shielding portions of inner tip conductors of the leads (also not separately shown in FIG. 1). In FIG. 1, only two leads are shown, a right ventricular (RV) lead and a left ventricular (LV) lead. A more complete lead system is illustrated in FIG. 15, described below. As will be explained further, ring electrode inductor elements may instead be positioned elsewhere along the lead, such as in the header of the lead, or may be positioned within the pacer/ICD itself, such as within the feed-through of the pacer/ICD. In some implementations, multiple inductor elements may be provided per lead, including additional tip electrode inductor elements.

As to the MRI system 18, the system includes a static field generator 20 for generating a static magnetic field 22 and a pulsed gradient field generator 24 for selectively generating pulsed gradient magnetic fields 26. The MRI system also includes an RF generator 28 for generating pulsed RF fields 27. Other components of the MRI, such as its sensing and imaging components are not shown either. MRI systems and imaging techniques are well known and will not be described in detail herein. For exemplary MRI systems see, for example, U.S. Pat. No. 5,063,348 to Kuhara et al., entitled “Magnetic Resonance Imaging System” and U.S. Pat. No. 4,746,864 to Satoh, entitled “Magnetic Resonance Imaging System.” Note that the fields shown in FIG. 1 are stylized representations of the MRI fields intended merely to illustrate the presence of the fields. Actual MRI fields generally have far more complex patterns.

The pacer/ICD 10 senses cardiac electrical signals via the electrodes on the leads and administers electrotherapy to the cardiac tissue via the electrodes on the leads.

With reference to the remaining figures, the band stop filter systems and methods will be explained in greater detail with reference to various illustrative examples.

Leads with Inductor Elements (Band Stop Filters) to Reduce MRI-Induced Heating

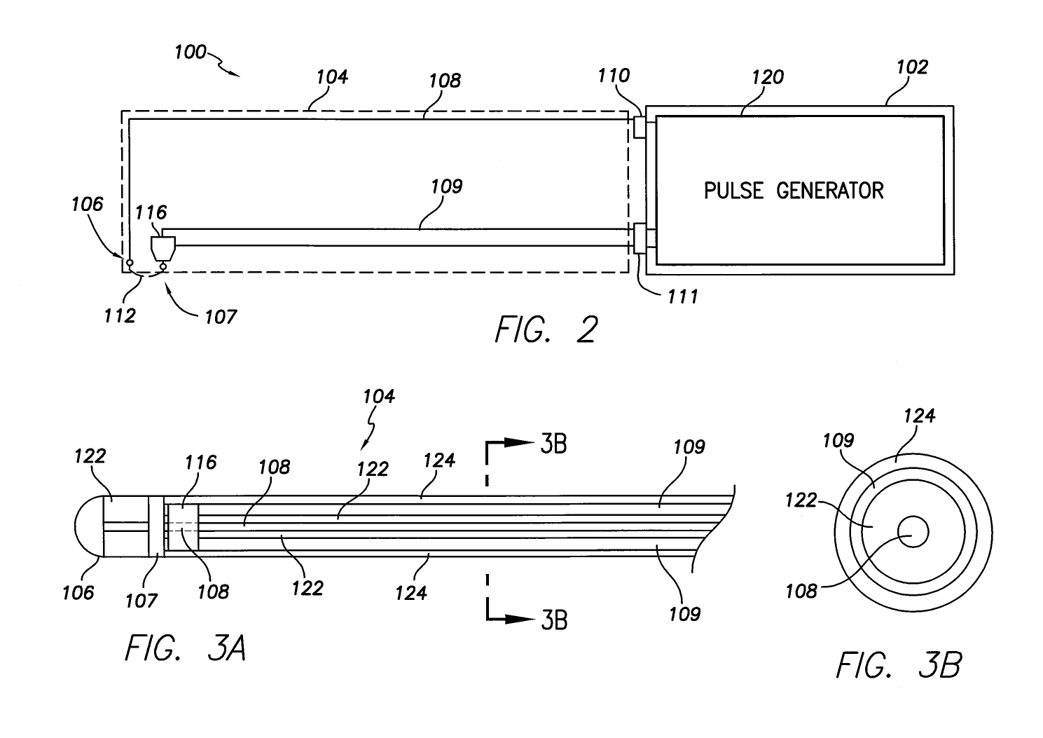

FIG. 2 illustrates an implantable system 100 having a pacer/ICD or other implantable medical device 102 with a bipolar coaxial lead 104. The bipolar lead includes a tip electrode 106 connected to the pacer/ICD via a tip conductor 108 coupled to a tip connector or terminal 110 of the pacer/ICD. The bipolar lead also includes a ring electrode 107 connected to the pacer/ICD via a ring conductor 109 coupled to a ring connector or terminal 111 of the pacer/ICD. Depending upon the particular implementation, during pacing/sensing, the tip electrode may be more negative than the ring, or vice versa. A conducting path 112 between the tip electrode 106 to the ring electrode 107 is provided through patient tissue (typically cardiac tissue.) A ring inductive element or other band stop filter 116 is positioned along conductor 109 at a distal portion thereof near the ring electrode 107, principally to reduce tip heating, though it also helps to reduce any ring heating. The ring inductive element 116 is configured such that it acts at a band stop filter at the RF of the MRI. For example, the inductive element is configured to act as a band stop filter at 64 MHz, the RF of a 1.5 T MRI and/or at 128 MHz, the RF of a 3.0 T MRI. In some embodiments, the inductive element 116 may configured to act as a band stop filter with self-resonant frequencies at the RF of an MRI as follows: 63.7 MHz+−0.345 MHz for 1.5 T or 125.6+−3.5 MHz for 3 T.

Thus, when the inductive element 116 is present in the magnetic field of the MRI, the RF of the MRI causes high impedance at the element 116, substantially reducing, if not totally eliminating, induced currents in the ring conductor 109 and the ring electrode 107. In other words, the band stop filter 116 is provided to block signals at the RF frequencies of MRI fields. At RF ranges other than the RF of the MRI, the inductive element 116 is generally invisible to the circuit including the ring conductor 109 and the ring electrode 107. As a result, the inductive element 116 does not adversely impact the therapeutic and sensing operation of the circuit including the ring conductor and the ring electrode. The band stop filter 116 may be implemented using any suitable technology such as coil inductors, integrated circuit (IC) inductors (i.e. printed traces on multi-layers), LC resonant tanks, etc.

As shown, the pacer/ICD includes a pulse generator 120 for generating therapeutic pacing pulses for delivery to patient tissue via the tip and ring electrodes in accordance with conventional pacing techniques. Note that the pacer/ICD may include a wide variety of other components for controlling pacing/sensing/shocking.

With the coaxial lead arrangement of FIG. 2, during an MRI, a current loop might be induced via the MRI RF within the lead (and within circuit components within the pacer/ICD that electrically connect terminals 110 and 111) if no inductor element 116 were present. Without the band stop filter characteristics provided by the inductor element, the MRI RF induced current loop might pass through patient tissue from the tip electrode to the ring electrode before returning to the pacer/ICD, causing considerable resistive heating at the tip electrode and in the intervening tissue. As explained above, such heating can damage patient tissue and interfere with pacing and sensing. With the band stop filter characteristics of the inductor element 116, the MRI RF cannot induce the current loop through the circuit including the inductor element 116, thereby blocking a significant source of tip heating. Note, though, that current loops might potentially still be MRI RF induced that pass from the tip electrode to the housing of the pacer/ICD or to other electrodes within the lead system, such as the tip electrodes of other nearby leads. However, by providing the band stop filtering at the MRI RF via the inductor element 116 in the circuit including the ring conductor 109 and ring electrode 107, the ring conductor 109 acts as a floating coil at the MRI RF and an RF shield to shield a large portion of the inner, tip conductor, thus reducing the likelihood of currents being induced via the tip conductor, the tip electrode, and other electrodes of the implanted system. This is illustrated more clearly in FIGS. 3A and 3B.

FIG. 3A illustrates a portion of bipolar lead 104, particularly illustrating the locations of tip electrode 106, ring electrode 107 and ring inductor element 116, as well as the coaxial configuration of the tip and ring conductors 108 and 109. As shown in FIG. 3B, Ring conductor 109 surrounds tip conductor 108 and separated therefrom by an insulator 122. An exterior surface of ring conductor 109 is covered by or coated by another insulator 124. With this arrangement, when inductor element 116 is acting as a band stop filter for the RF of the MRI, ring conductor 109 can be considered to be a floating outer coil 109. Since the ring conductor extends the length of the lead from ring electrode to the header of the lead (not specifically shown in FIGS. 2 and 3A), the ring conductor thereby covers a substantial portion of the inner tip conductor 108 and acts as an RF shield to those portions of the tip conductor during an MRI procedure. Hence, any currents that would otherwise be induced along the tip conductor by the RF fields of the MRI are substantially reduced.

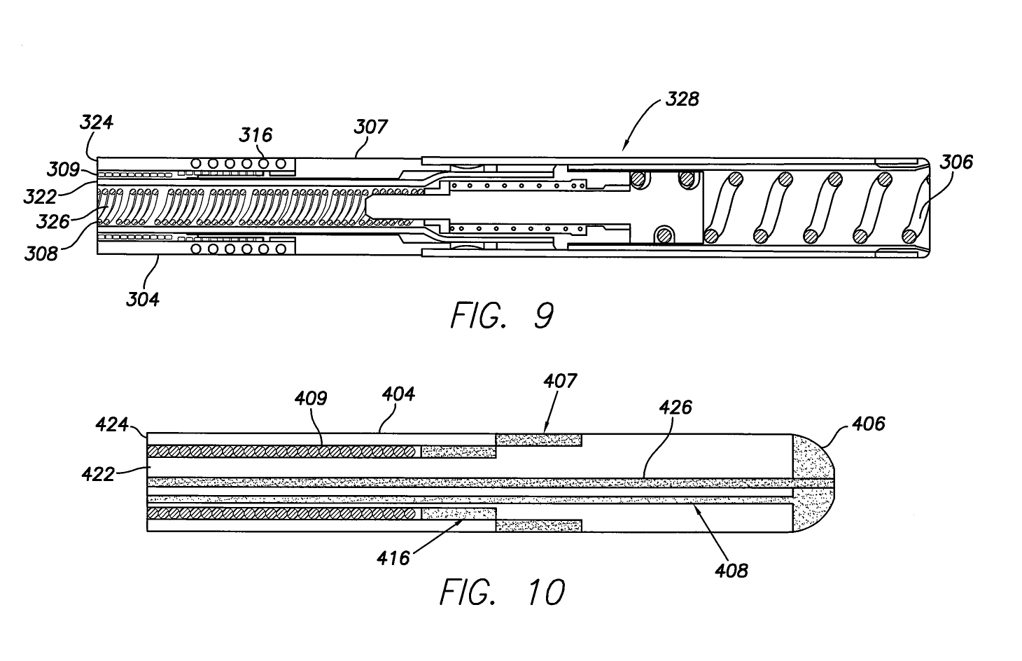

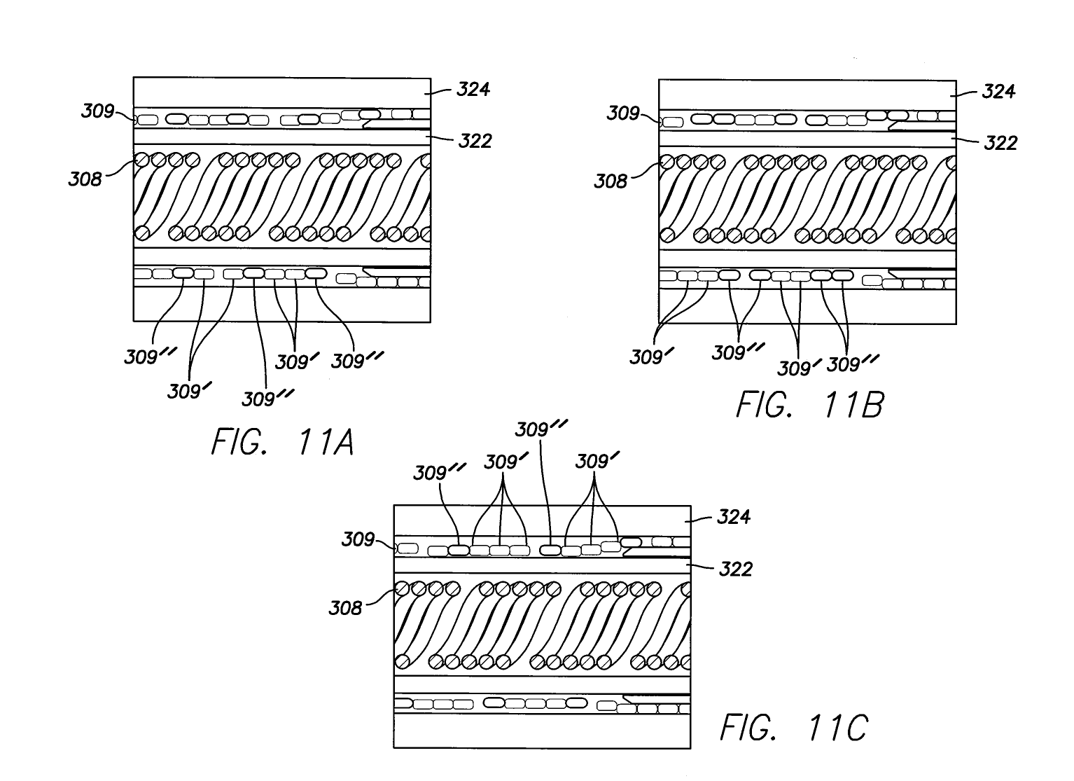

Depending upon the particular implementation, the RF shielding provided by ring conductor 109 may be sufficient to reduce induced currents along tip conductor 108 by an amount sufficient to prevent any significant tip heating, such that a separate tip inductor element is not needed. In other implementations, to be discussed below, the RF shielding provided by the ring conductor is at least sufficient to reduce the induced voltages within tip conductor to permit the use of a physically smaller and less robust inductor element along the tip conductor (see FIG. 9).

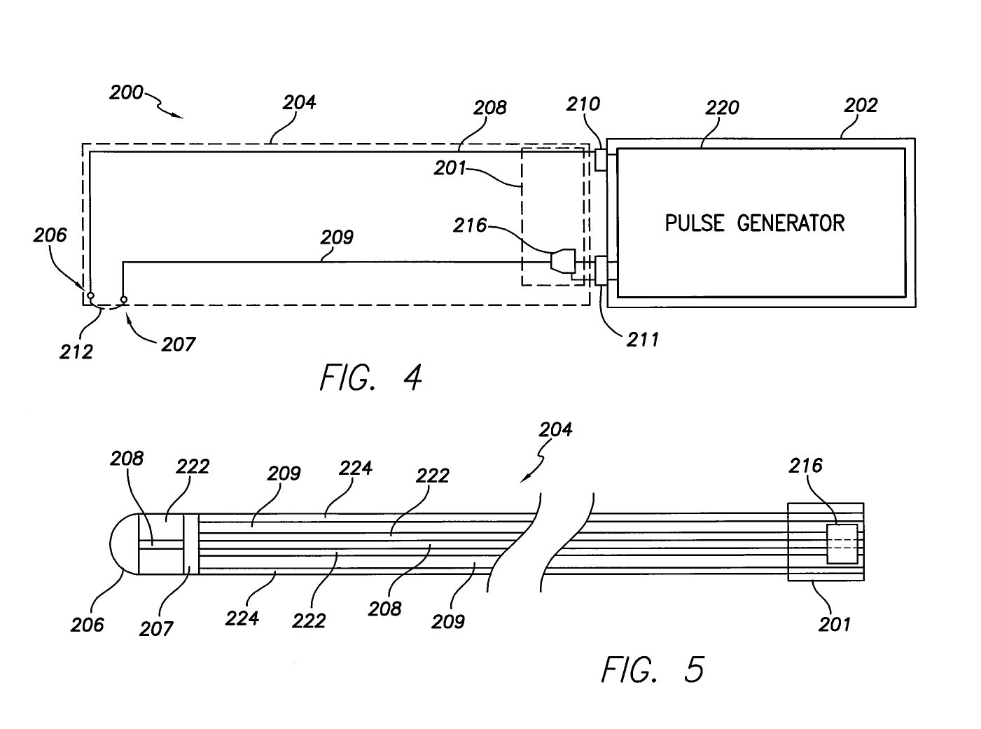

FIGS. 4 and 5 illustrate an alternative implantable medical system 200 wherein the ring electrode inductor element 216 is mounted within a header 201 of a bipolar coaxial lead 204, which is connected to a pacer/ICD 202. Locating the inductor element 216 outside, but adjacent to, the feedthru of the pacer/ICD acts to prevent RF from entering the pacer/ICD. Again, the bipolar lead includes a tip electrode 206 connected to the pacer/ICD via a tip conductor 208, which is in turn coupled to a tip terminal 210 of the pacer/ICD. The bipolar lead also includes ring electrode 207 connected via ring conductor 209 coupled to ring terminal 211. A conducting path 212 is provided through patient tissue from the tip electrode to the ring electrode. The ring inductor element or other band stop filter 216 is positioned at or near a proximal end of conductor 209 within header 201.

FIG. 5 illustrates a portion of bipolar lead 204, particularly illustrating the locations of tip electrode 206, ring electrode 207 and ring inductor element 216, as well as the coaxial configuration of the tip and ring conductors 208 and 209. Ring conductor 209 surrounds tip conductor 208 and is separated therefrom by an insulator 222. An exterior surface of ring conductor 209 is covered by insulator 224.

The inductor element 216 is provided primarily to reduce tip heating, though it also helps to reduce any ring heating. The ring inductive element 216 is configured such that it acts at a band stop filter at the RF of the MRI. For example, the inductive element is configured to act as a band stop filter at 64 MHz, the RF of a 1.5 T MRI and/or at 128 MHz, the RF of a 3.0 T MRI. Thus, when the inductive element 216 is present in the magnetic field of the MRI, the RF of the MRI causes high impedance at the element 216, substantially reducing, if not totally eliminating, induced currents in the ring conductor 209 and the ring electrode 207. At RF ranges other than the RF of the MRI, the inductive element 216 is generally invisible to the circuit including the ring conductor 209 and the ring electrode 207. As a result, the inductive element 216 does not adversely impact the therapeutic and sensing operation of the circuit including the ring conductor and the ring electrode.

As discussed above with respect to FIGS. 2, 3A and 3B, by providing the band stop filtering at the MRI RF via the inductor element 216 in the circuit including the ring conductor 209 and ring electrode 207, the ring conductor 209 acts as a floating coil at the MRI RF and an RF shield to shield a large portion of the inner, tip conductor. As a result, there is a reduced likelihood of currents being induced via the tip conductor, the tip electrode, and other electrodes of the implanted system.

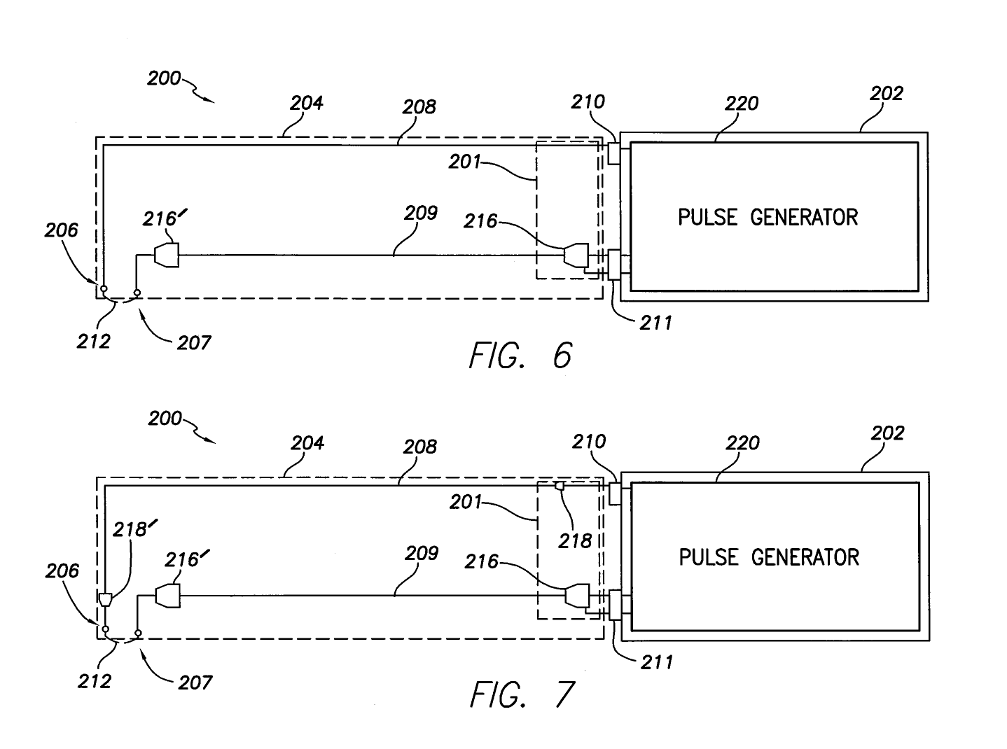

As indicated in FIG. 6, in some embodiments, implantable medical system 200 is configured as described above with respect to FIGS. 4 and 5, except ring inductor elements 216 and 216′ are respectively located at the proximal and distal ends of the ring conductor 209. Specifically, a proximal inductor element 216 is mounted on the ring conductor 209 within the header 201 of the bipolar coaxial lead 204, and a distal inductor element 216′ is mounted on the ring conductor 209 near the ring electrode 207. Such an embodiment will provide the band stop and floating coil (shielding) benefits discussed above with respect to FIGS. 4 and 5.

The embodiments disclosed above with respect to FIGS. 2-6 are advantageous in that no tip inductor is present and, as a result, no tip inductor heating exists, negating the need for a heat spreader (e.g., a Ti sleeve). Also, some of the embodiments provide more mechanical reliability; if the ring inductor fails, pacing/sensing from the tip electrode can still be done through unipolar pacing/sensing. Also, the absence of a tip inductor allows the mechanical design of the helical anchor header assembly of active fixation leads as discussed below with respect to FIG. 9 to be a very reliable and accepted configuration. Furthermore, the embodiments disclosed herein can be applied to co-axial, coil-cable mixed lead structures and active and passive fixation leads.

As indicated in FIG. 7, in some embodiments, implantable medical system 200 is configured as described above with respect to FIG. 6, except tip inductor elements 218 and 218′ are respectively located at the proximal and distal ends of the tip conductor 208. Specifically, a proximal inductor element 218 is mounted on the tip conductor 208 within the header 201 of the bipolar coaxial lead 204, and a distal inductor element 218′ is mounted on the tip conductor 208 near the tip electrode 206. Such an embodiment will provide the band stop and floating coil (shielding) benefits discussed above with respect to FIGS. 4 and 5.

In those embodiments having multiple inductive elements on a single conductor, as depicted in FIGS. 6 and 7, one of the inductive elements on a specific conductor may be configured to have a self-resonant frequency at a first frequency and the other of the inductive elements on the specific conductor may be configured to have a self-resonant frequency at a second frequency. For example, as can be understood from FIG. 7, the proximal inductor elements 216 and 218 may be configured to self-resonate 64 MHz to act as a band stop filter at 64 MHz, and the distal inductor elements 216′ and 218′ may be configured to self-resonate 128 MHz to act as a band stop filter at 128 MHz.

In some embodiments, the inductor elements 218 and 218′ of the tip conductor 208 may be less robust due to the presence of the inductor elements 216 and 216′ on the ring conductor 209. While the embodiments in FIG. 7 depicts two inductor elements on each conductor 208 and 209, in other embodiments the tip conductor 208 and/or the ring conductor 209 may each have only a single inductor element mounted.

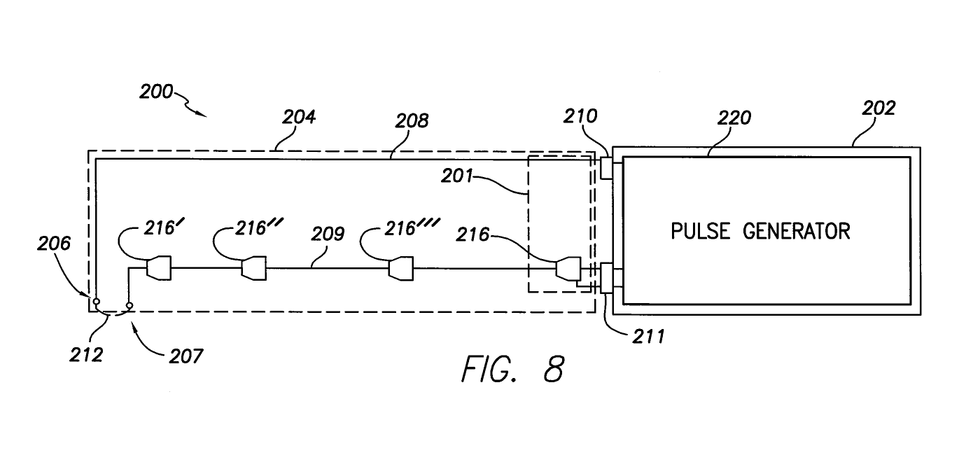

As can be understood from FIG. 8, in some embodiments, a conductor 209 may have more than two inductor elements mounted thereon. For example, inductor elements 216, 216′, 216″ and 216′″ may be located on ring conductor 209 and spaced at generally regular intervals, such as, for example, at quarter wavelengths.

In summary of the embodiments depicted in FIGS. 2-8, the pacer/ICD includes a pulse generator for generating therapeutic pacing pulses for delivery to patient tissue via the tip and ring electrodes in accordance with otherwise conventional pacing techniques when MRI fields are not present. During an MRI, a current loop might be induced within the lead if the band stop filter were not present. Without the band stop filter, the current loop might pass through patient tissue from the tip electrode to the ring electrode before returning to the pacer/ICD, causing considerable resistive heating at the tip electrode and in the intervening tissue. With the band stop filter, however, no RF current loops can pass through the band stop filter, thereby blocking a significant source of tip heating. Moreover, at RF frequencies, the ring conductor acts as an RF shield to shield a large portion of the inner, tip conductor, thus reducing the likelihood of currents being induced via the tip conductor, the tip electrode, and other electrodes of the implanted system.

FIG. 9 illustrates a longitudinal cross section of a distal end of an active fixation lead 304. The lead 304 includes a helical anchor electrode 306 extendable from the distal end of the lead and electrically coupled to a distal end of a helically wound tip conductor 308. A ring electrode 307 is proximally offset from the distal end of the lead and is electrically coupled to a helically wound ring conductor 309, which is located about the tip conductor 308 in a coaxial arrangement. The ring conductor 309 is sandwiched between an inner polymer insulation layer 322 and an outer polymer insulation layer 324, which are arranged in a coaxial arrangement. The inner layer 322 separates the conductors 308 and 309 from each other, and the outer layer 324 defines an outer circumferential surface of the lead. The tip conductor 308 defines a central lumen 326

The inductor locations discussed above with respect to FIGS. 2-8 may be employed in an active fixation lead 304 as depicted in FIG. 9. For example, an inductor element 316 as discussed above may be located in the outer layer 324 immediately proximal the proximal edge of the ring electrode 307. As mentioned above with respect to FIG. 2, providing an inductor element on the ring conductor (with no such inductor element on the tip conductor) may be sufficient to address any RF induced current issues in the tip inductor due to the ring conductor acting as a shield. Thus, in such an embodiment, a standard header assembly 328 may be employed for the helical anchor electrode, as depicted in FIG. 9.

FIG. 10 illustrates a longitudinal cross section of a distal end of a passive fixation lead 404. The lead 404 includes a tip electrode 406 forming the distal end of the lead and electrically coupled to a distal end of a tip conductor 408, which may be helically routed as depicted in FIG. 9 or a linearly routed cable as depicted in FIG. 10. A ring electrode 407 is proximally offset from the distal end of the lead and is electrically coupled to a helically wound ring conductor 409, which is located about the tip conductor 408. The ring conductor 409 is sandwiched between an inner polymer insulation layer 422 and an outer polymer insulation layer 424, which are arranged in a coaxial arrangement. The tip conductor 408, when in the form of a linearly routed cable, extends through the inner layer 422, which separates the conductors 408 and 409 from each other. The outer layer 424 defines an outer circumferential surface of the lead. A central lumen 426 extends through the lead adjacent to the tip conductor 408.

The inductor locations discussed above with respect to FIGS. 2-8 may be employed in a non-coaxial type lead, such as a Tachy lead, CRT lead or passive fixation lead 404 as depicted in FIG. 10. For example, an inductor element 416 as discussed above may be located in the outer layer 424 immediately proximal the proximal edge of the ring electrode 407. As mentioned above with respect to FIG. 2, providing an inductor element on the ring conductor (with no such inductor element on the tip conductor) may be sufficient to address any RF induced current issues in the tip inductor due to the ring conductor acting as a shield.

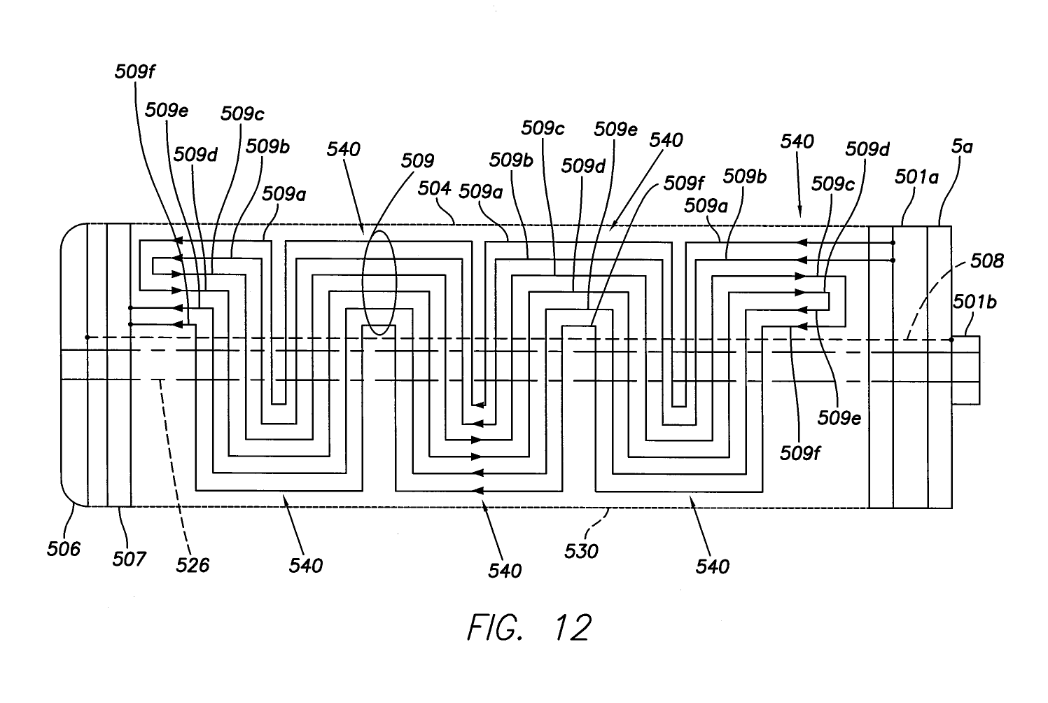

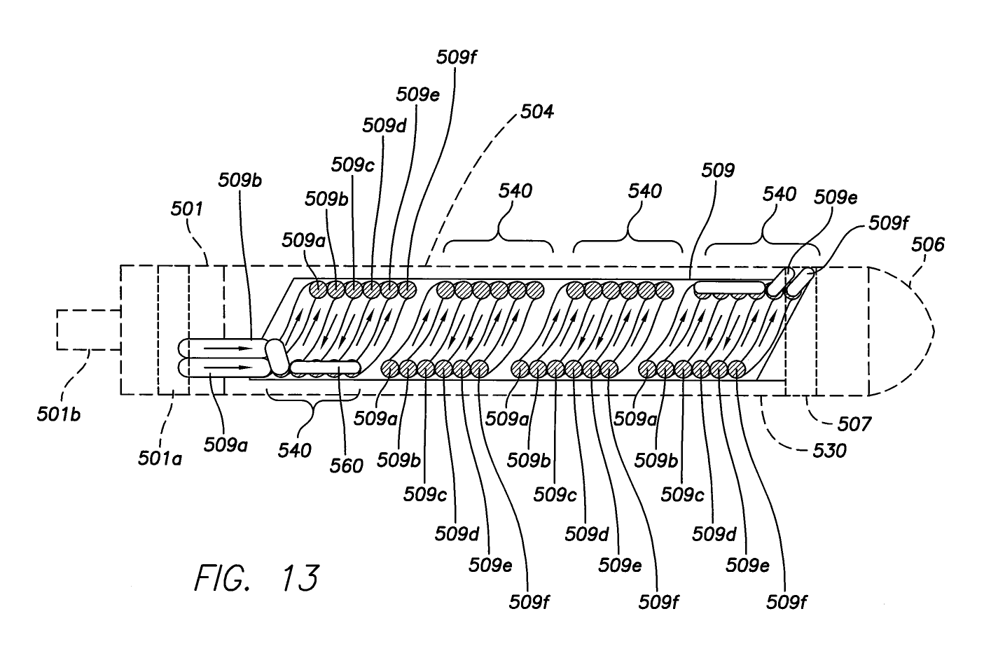

As shown in FIGS. 11A-11C, which are longitudinal cross section segments of a lead body similar to that depicted in FIG. 9, the ring conductor 309 is sandwiched between insulation layers 322 and 324. In some embodiments, the insulation layers 322 and 324 may be or include shrink tubing. In other words, the insulation layers 322 and 324 may be or include shrink tubing 322 and 324 above and below the layers of the ring conductor 309, the shrink tubing acting to electrically insulate the ring conductor 309 from fluid outside or inside the lead body.

As a comparison of FIGS. 11A-11C indicates, in some embodiments, the filars of the multi-filar helically wound coil forming the ring conductor 309 may have a variety of insulation patterns, which may facilitate a reduction in RF induced currents by lengthening the current pathway along the ring conductor. For example, as shown in FIG. 11A, in one embodiment, the filars of the ring conductor 309 may have a pattern of two un-insulated filars 309′ to one insulated filar 309″. As indicated in FIG. 11B, in another embodiment, the filars of the ring conductor 309 may have a pattern of two un-insulated filars 309′to two insulated filars 309″. As illustrated in FIG. 11C, in another embodiment, the filars of the ring conductor 309 may have a pattern of three un-insulated filars 309′ to one insulated filars 309″. Other ratios between un-insulated and insulated filars are possible, including, for example: 3:2; 3:3; 4:1; 4:2; etc. The pattern of multi-filars can be configured as needed during manufacturing to obtain a desired resistance and also meet pacing/sensing requirements.

The insulation on the insulated filars 309″ may be ETFE or another dielectric material. The shrink tubing over and under the ring conductor 309 may adhere to the outer and inner circumferential surfaces of the ring conductor to ensure the current does not short around the insulated filars 309″ between un-insulated filars 309′, undesirably shortening the current pathway along the ring conductor 309.

In some embodiments, all of the filars 309 are individually electrically insulated. For example, each of the filars 309 may have its own electrically insulating jacket. As a result, all of the filars 309 are electrically insulated from each other.

In some embodiments, the tip conductor 308 may employ a similar insulation pattern and material as discussed above with respect to FIGS. 11A-11C for the ring conductor 309. Insulating the filars of the conductor in the patterns discussed above has the impact of lengthening the current path along the conductor between an electrode and its respective electrical contact on the lead connector end of the lead. The resulting higher resistance inside the conductor can be used to block RF induced currents.

In some embodiments, the ring conductor 309 and/or the tip conductor 308 are formed of filars with conductive cores formed of a high resistive metal that meets DCR of at least approximately 60 ohms, but less than approximately 80 ohms for pacing/sensing requirements. In some embodiments, the filar cores are formed of MP35N or another metal having a similar resistance. Such MP35N filars may be insulated as discussed above with respect to FIGS. 11A-11C.

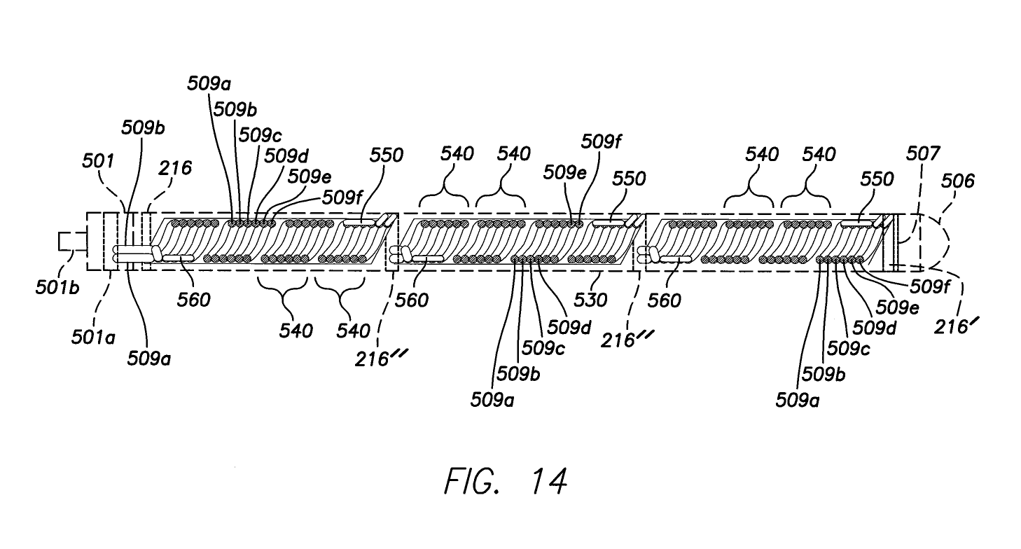

As will now be discussed with respect to FIG. 12, which is a schematic diagram of a lead, and FIG. 13, which is a longitudinal cross section of a lead ring conductor with the rest of the lead depicted in phantom lines, there is another way of increasing the length of the current pathways through the lead. As shown in FIGS. 12 and 13, the lead 504 includes a tip electrode 506 and a ring electrode 507 at the distal end of the lead and a lead connector end 501 at the proximal end of the lead. The lead connector end 501 includes a ring contact 501a and a pin contact 501b. A lumen 526 extends through the lead body 530 from the pin contact 501b to the tip electrode 506. A tip conductor 508 (shown in FIG. 12, but not FIG. 13) extends from the pin contact 501b to the tip electrode 506 and may be in the form of a helically wound multi-filar coil, a solid wire or a multi-filar cable. A ring conductor 509 in the form of a multi-filar (e.g., six filar) helically wound coil extends from the ring contact 501a to the ring electrode 507. Each filar 509a-509f of the multi-filar ring conductor 509 is individually insulated from its immediately adjacent neighbor filars. In one embodiment, each filar 509a-509f has its own dielectric insulation jacket to electrically isolate each filar 509a-509f from it neighbor filars.

The six filars 509a-509f forming the ring conductor 509 are helically wound such that each coil or loop 540 of the ring conductor 509 includes a coil or loop of each of the filars 509a-509f. As indicated in FIGS. 12 and 13, filars 509a and 509b extend into the lead connector end 501 to electrically couple to the ring contact 501a. The filars 509a and 509b helically extend through the ring conductor 509 to the distal end of the ring conductor as the first pair of filars 509a and 509b. At the distal end of the lead, the first pair of filars 509a and 509b is electrically coupled via an electrical connection 550 to the second pair of filars 509c and 509d of the ring conductor 509. The second pair of filars 509c and 509d helically extends through the ring conductor 509 to the proximal end of the ring conductor. At the proximal end of the lead, the second pair of filars 509c and 509d is electrically coupled via an electrical connection 560 to the third pair of filars 509e and 509f of the ring conductor 509. The third pair of filars 509e and 509f helically extends through the ring conductor 509 to the distal end of the ring conductor to electrically couple to the ring electrode 507. Thus, as can be understood from the arrows on the filars 509a-509f of FIGS. 12 and 13, because each filar 509a-509f is individually electrically insulated and electrically isolated from its neighbor filars 509a-509f and the three pairs of filars are electrically coupled with each other such that the result is an overall pair of filars that extends proximal to distal followed by distal to proximal followed by proximal to distal, electrical current (represented by the arrows on the filars 509a-509f) is forced to travel the length of the ring conductor 509 three times when flowing from the ring contact 501a to the ring electrode 507. Specifically, the electrical current travels from the ring contact 501a to the first pair of filars 509a and 509b, distally along the first pair of filars 509a and 509b to the proximal electrical connection 550 and into the second pair of filars 509c and 509d, proximally along the second pair of filars 509c and 509d to the distal electrical connection 560 and into the third pair of filars 509e and 509f, and distally along the third pair of filars 509e and 509f and into the ring electrode 507. Thus, the filars of the coil can be said to double back on themselves or form a double back type of pattern such that the current is caused to double back on itself three times in its travel along the filars between the lead connector end and the ring electrode.

While the embodiment discussed with respect to FIGS. 12 and 13 is given in the context of the ring conductor 507, in some embodiments, the tip conductor 506 will be the conductor so configured. In other embodiments, both the ring conductor 507 and the tip conductor 506 can be configured as discussed with respect to FIGS. 12 and 13.

In one embodiment, for example, a lead has a six filar inner coil (e.g., a tip conductor 508) and the first pair of filars are electrically connected at the proximal end to the pin contact 501b and each other, but are electrically isolated from the second and third pairs of filars. At the distal end the first pair of filars are electrically connected to the second pair of filars, but electrically isolated from the third pair of filars. At the proximal end the second pair of filars are electrically connected to the third pair of filars, but electrically isolated from the first pair of filars. At the distal end the third pair of filars are electrically coupled to the tip electrode 506. Thus, in a manner similar to that of the ring conductor 509 discussed with respect to FIGS. 12 and 13, the total length of current path from the pin contact 501b to the tip electrode 506 across the tip conductor 508 would be three times what it would otherwise be were the filars not individually electrically insulated and the current could jump directly from filar coil to filar coil instead of being forced to track the entire length of the tip conductor three times. Because the RF induced currents are caused to flow in opposite directions between adjacent filar pairs, induced currents are reduced. Also, the tripled length of the current path causes an increase in resistance and a reduction of RF induced currents.

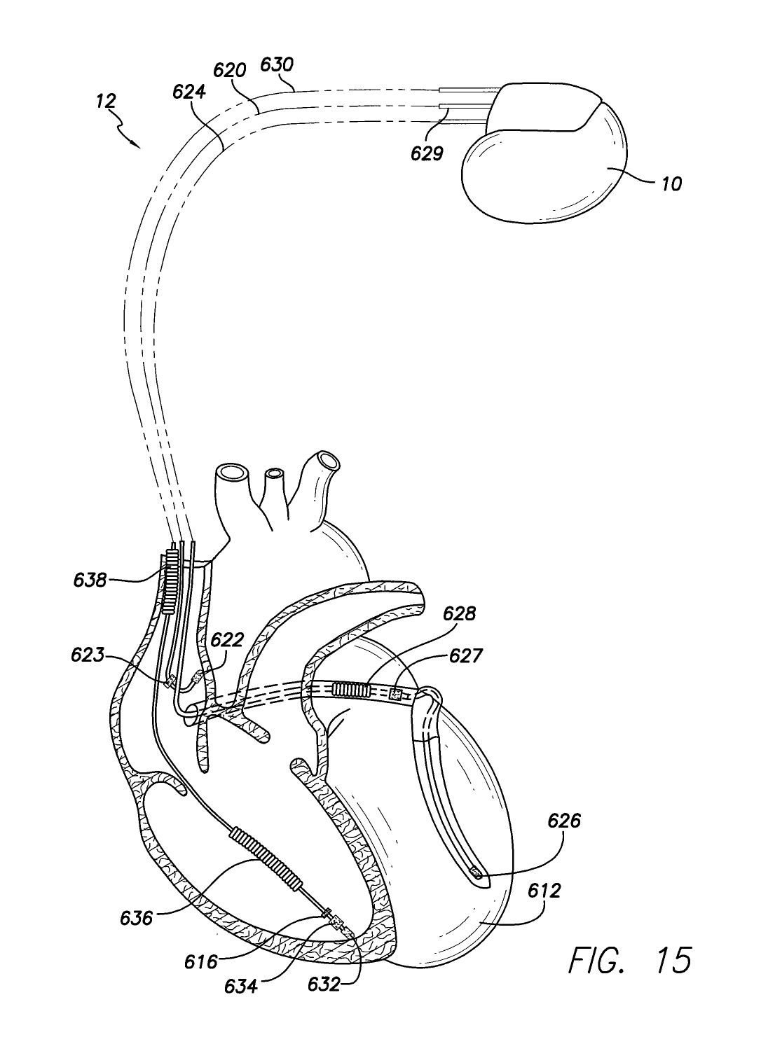

In one embodiment, the zig-zag wiring arrangement discussed above with respect to FIGS. 12 and 13 can be employed between inductor element locations. For example, as can be understood from FIG. 14, where the concepts illustrated in FIGS. 8, 12 and 13 are combined, the lead employs multiple inductor elements 216, 216′, 216″, 216′″ at a spacing of, for example, a quarter wavelength (as shown in FIGS. 8 and 14), and the zig-zag wiring arrangement of the filars 509a-509f is implemented between each pair of adjacent inductor elements 216, 216′, 216″, 216′″ in a manner similar to that implemented between the ring contact and the ring electrode in FIGS. 12 and 13.

The various systems and methods described above can be exploited for use with a wide variety of implantable medical systems. For the sake of completeness, a detailed description of an exemplary pacer/ICD and lead system will now be provided.

While the embodiments discussed above with respect to FIG. 12-14 are given in the context of the ring conductor 509 being a helically wound multi-filar coil conductor configured to have the zig-zag wiring arrangement discussed above, in other embodiments, the ring conductor 509 may be a helically wound or twisted multi-filar cable configured to have the zig-zag wiring arrangement discussed above. In such a cable configuration, each of the filars is individually insulated from its immediately adjacent neighbor filars. In one embodiment, each filar has its own dielectric insulation jacket to electrically isolate each filar of the cable from it neighbor filars.

Similar to as depicted in FIG. 12, the distal ends of a first group of one or more filars of the cable that are electrically coupled to the ring contact 507 are electrically coupled to the distal ends of a second group of one or more filars of the cable that extend proximally. The proximal ends of the second group of one or more filars of the cable are electrically coupled to the proximal ends of a third group of one or more filars of the cable that extend distally to electrically couple to the ring electrode 501a. Thus, as with the above described zig-zag arrangement for the helically wound coil conductor, a zig-zag arrangement may be formed for a helically twisted multi-filar cable conductor, thereby increasing the length the current has to travel through the cable and causing the current to make a zig-zag trip through the cable.

### Exemplary Pacer/ICD/Lead System

FIG. 15 provides a simplified diagram of the pacer/ICD of FIG. 1, which is a dual-chamber stimulation device capable of treating both fast and slow arrhythmias with stimulation therapy, including cardioversion, defibrillation, and pacing stimulation. To provide atrial chamber pacing stimulation and sensing, pacer/ICD 10 is shown in electrical communication with a heart 612 by way of a left atrial lead 620 having an atrial tip electrode 622 and an atrial ring electrode 623 implanted in the atrial appendage. Pacer/ICD 10 is also in electrical communication with the heart by way of a right ventricular lead 630 having, in this embodiment, a ventricular tip electrode 632, a right ventricular ring electrode 634, a right ventricular (RV) coil electrode 636, and a superior vena cava (SVC) coil electrode 638. Typically, the right ventricular lead 630 is transvenously inserted into the heart so as to place the RV coil electrode 636 in the right ventricular apex, and the SVC coil electrode 638 in the superior vena cava. Accordingly, the right ventricular lead is capable of receiving cardiac signals, and delivering stimulation in the form of pacing and shock therapy to the right ventricle. A ring inductor element or band stop filter 616, configured as described above, is positioned near ring electrode 634. In the figure, the ring inductor element is shown in phantom lines, as it is internal to the lead.

To sense left atrial and ventricular cardiac signals and to provide left chamber pacing therapy, pacer/ICD 10 is coupled to a “coronary sinus” lead 624 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 624 is designed to receive atrial and ventricular cardiac signals and to deliver left ventricular pacing therapy using at least a left ventricular tip electrode 626, left atrial pacing therapy using at least a left atrial ring electrode 627, and shocking therapy using at least a left atrial coil electrode 628. With this configuration, biventricular pacing can be performed. Although only three leads are shown in FIG. 15, it should also be understood that additional stimulation leads (with one or more pacing, sensing and/or shocking electrodes) may be used in order to efficiently and effectively provide pacing stimulation to the left side of the heart or atrial cardioversion and/or defibrillation. Also, additional ring inductor or band stop filters may be installed in the various leads, as already explained, such as in the LV/CS lead or the RA lead. Ring inductor or band stop filters may be installed at other locations within the leads, such as within lead headers 629. Also, tip inductor or band stop filters may be installed.

What have been described are systems and methods for use with a set of pacing/sensing leads for use with a pacer/ICD. Principles of the invention may be exploiting using other implantable systems or in accordance with other techniques. Thus, while the invention has been described with reference to particular exemplary embodiments, modifications can be made thereto without departing from the scope of the invention.

What is claimed is:

1. An implantable medical lead comprising:
a tubular body including a proximal end and a distal end;
an electrode coupled to the body near the distal end;
a lead connector end coupled to the body near the proximal end; and
a helical conductor coil extending through the body from the lead connector end to the electrode,
wherein, in extending through the body, the helical conductor coil first extends distally for a distance, then proximally for the distance, and then distally for the distance within a single wound layer of the helical conductor coil.

2. The lead of claim 1, wherein the electrode includes a ring electrode.

3. The lead of claim 1, wherein the helical conductor coil includes multiple filars.

4. The lead of claim 3, wherein, in the helical conductor coil first extending distally for a distance, then proximally for the distance, and then distally for the distance, the multiple filars first extend distally for the distance, then proximally for the distance, and then distally for the distance, the filars forming a double back type of pattern within the single wound layer of the helical conductor coil.

5. The lead of claim 4, further comprising at least one band stop filter coupled to the helical conductor coil and located between the lead connector end and the electrode, wherein the filars form a first double back pattern between the lead connector end and the band stop filter and a second double back pattern between the band stop filter and the electrode.

6. The lead of claim 5, wherein the band stop filter includes at least one of a coil inductor, an integrated circuit inductor, or a LC resonant tank.

7. The lead of claim 4, further comprising multiple band stop filters coupled to the helical conductor coil and located between the lead connector end and the electrode, wherein the filars form a double back pattern between a pair of band stop filters.

8. The lead of claim 7, wherein the band stop filters are spaced apart from each other along the tubular body at a distance of approximately a quarter wavelength.

9. The lead of claim 3, wherein the multiple filars includes a first number of filars, a second number of filars and a third number of filars, and wherein, in the helical conductor coil first extending distally for a distance, then proximally for the distance, and then distally for the distance, the first number of filars extending distally for the distance and join to a distal end of the second number of filars, the second number of filars extending proximally for the distance and join to a proximal end of the third number of filars, the third number of filars extending distally, the filars forming a double back type of pattern within the single wound layer of the helical conductor coil.

10. The lead of claim 3, wherein the first number of filars is one filar.

11. The lead of claim 3, wherein the first number of filars is a pair of filars.

12. The lead of claim 3, wherein at least one of the filars of the multiple filars are insulated and at least another of the filars of the multiple filars are uninsulated, there being a pattern of uninsulated to insulated filars in the helical conductor coil.

13. The lead of claim 12, wherein the pattern of uninsulated to insulated filars includes at least one of: 2 to 1; 2 to 2; 3 to 1; 3 to 2; 3 to 3; 4 to 1; or 4 to 2.

14. The lead of claim 1, wherein each filar has an individual electrical insulation jacket.

15. The lead of claim 12, wherein at least some of the filars include electrically conductive portions formed of MP35N.

16. An implantable medical lead comprising:
a tubular body including a proximal end and a distal end;
an electrode coupled to the body near the distal end;
a lead connector end coupled to the body near the proximal end; and
a helical conductor coil extending through the body from the lead connector end to the electrode and being configured to cause current to double back along at least a portion of the length of the helical conductor coil within a single wound layer of the helical conductor coil.

17. The lead of claim 16, further comprising a pair of band stop filters, the current doubling back between the pair of band stop filters.

18. The lead of claim 17, wherein the band stop filters are spaced apart from each other at approximately a quarter wavelength.

19. The lead of claim 16, wherein at least one of the band stop filters includes at least one of a coil inductor, an integrated circuit inductor, or a LC resonant tank.

20. The lead of claim 16, wherein the helical conductor comprises a helically twisted multi-filar conductor cable.

21. A method of assembling an implantable medical lead, the method comprising:
helically co-winding multiple filars into a single helical layer;
electrically joining a distal end of a first group of one or more of the multiple filars to a distal end of a second group of one or more of the multiple filars;
electrically joining a proximal end of the second group of one or more of the multiple filars to a proximal end of a third group of one or more of the multiple filars;
electrically coupling a proximal end of the first group of one or more of the multiple filars to an electrical contact of a lead connector end; and
electrically coupling a distal end of the third group of one or more of the multiple filars to a distal electrode.

22. The method of claim 21, wherein the single helical layer includes part of a helically wound multi-filar coil conductor.

23. The method of claim 21, wherein the single helical layer includes part of a helically wound multi-filar cable conductor.
